(19) United States
(12) Patent Application Publication
(10) Pub. No.: US 2020/0141914 A1
(43) Pub. Date: May 7, 2020

(54) GAS SENSOR AND CONSTANT-TEMPERATURE APPARATUS

(71) Applicant: PHC HOLDINGS CORPORATION, Tokyo (JP)

(72) Inventors: Kiminori MIZUUCHI, Osaka (JP); Masaki YAMAMOTO, Ehime (JP); Atsunobu FUWA, Kanagawa (JP)

(21) Appl. No.: 16/730,899

(22) Filed: Dec. 30, 2019

Related U.S. Application Data
(63) Continuation of application No. PCT/JP2018/019599, filed on May 22, 2018.

(30) Foreign Application Priority Data
Jul. 7, 2017 (JP) 2017-133950

(51) Int. Cl. G01N 33/00

(52) U.S. Cl. CPC G01N 33/0016; G01N 21/3504; G01N 33/0027

(57) ABSTRACT

A gas sensor includes: a light source that emits a first light of a predetermined wavelength toward a gas subject to detection; a density detector that receives the first light and detects a density of the gas subject to detection based on absorption of the first light by the gas subject to detection; a translucent member provided between the light source and the density detector; a temperature adjustment unit that varies a temperature of the translucent member; and a humidity detection unit that detects a humidity of the gas subject to detection based on variation in an amount of the first light received in the density detector and by using a temperature of the translucent member and an ambient temperature of the gas sensor.

### CROSS-REFERENCE TO RELATED APPLICATIONS

This application is based upon and claims the benefit of priority from the prior Japanese Patent Application No. 2017-133950, filed on Jul. 7, 2017 and International Patent Application No. PCT/JP2018/019599, filed on May 22, 2018, the entire content of each of which is incorporated herein by reference.

### BACKGROUND

### Field of the Invention

The present invention relates to a gas sensor and a constant-temperature apparatus provided with the gas sensor.

### Description of the Related Art

Gas sensors capable of detecting a gas such as CO₂ and NOx are known in the related art. By way of example of such a gas sensor, patent literature 1 discloses a gas sensor capable of detecting the density of a gas subject to detection by using absorption of infrared light by the gas subject to detection.

More specifically, the infrared gas sensor is provided with a light source, a light detection unit, a temperature measurement unit for measuring the temperature of the light detection unit, and a humidity measurement unit for measuring the humidity of a sealing unit for sealing the light detection unit based on the internal resistance of the light detection unit. The density of the gas subject to detection is calculated based on the output of the light detection unit, the temperature of the light detection unit, and the humidity of the sealing unit. In the gas sensor disclosed in patent literature 1, highly precise gas density detection is realized by correcting the output of the light detection unit based on the temperature information and the humidity information.
[patent literature 1] JP2017-15508

There is a requirement to detect the humidity, as well as the density, of the gas subject to detection by the gas sensor described above.

### SUMMARY OF THE INVENTION

The embodiments address the above-described issues, and a general purpose thereof is to provide a technology for detecting the density and humidity of the gas subject to detection.

An embodiment of the present disclosure is a gas sensor. A gas sensor includes: a light source that emits a first light of a predetermined wavelength toward a gas subject to detection; a density detector that receives the first light and detects a density of the gas subject to detection based on absorption of the first light by the gas subject to detection; a translucent member provided between the light source and the density detector; a temperature adjustment unit that varies a temperature of the translucent member; and a humidity detection unit that detects a humidity of the gas subject to detection based on a variation in an amount of the first light received in the density detector and by using a temperature of the translucent member and an ambient temperature of the gas sensor.

Another embodiment relates to a constant-temperature apparatus. The constant-temperature apparatus includes: a constant-temperature tank that houses a gas; and the gas sensor according to the above embodiment, wherein the gas sensor detects a density and humidity of the gas in the constant-temperature tank.

### BRIEF DESCRIPTION OF THE DRAWINGS

Embodiments will now be described, by way of example only, with reference to the accompanying drawings which are meant to be exemplary, not limiting, and wherein like elements are numbered alike in several Figures, in which:

FIG. 1 is a horizontal cross-sectional view showing a part of a constant-temperature apparatus according to embodiment 1;

FIG. 2A is a graph showing a temperature variation in the first translucent member and the second translucent member that occurs when the humidity is detected;

FIG. 2B is a graph showing a variation in the amount of light received by the density detector when the humidity is detected;

FIG. 3 is a flowchart showing the operation of the gas sensor according to embodiment 1;

FIG. 4 is a horizontal cross-sectional view schematically showing a part of the gas sensor according to variation 1;

FIG. 5 is a horizontal cross-sectional view schematically showing a part of the gas sensor according to variation 2;

FIG. 6A is a plan view schematically showing the density detector provided in the gas sensor according to embodiment 2;

FIG. 6B is a side view schematically showing the density detector provided in the gas sensor according to embodiment 2;

FIG. 7A is a plan view schematically showing the density detector provided in the gas sensor according to embodiment 3;

FIGS. 7B and 7C are side views schematically showing the density detector provided in the gas sensor according to embodiment 3;

FIG. 8 is a vertical cross-sectional view schematically showing the gas sensor according to embodiment 4;

FIG. 9A is a vertical cross-sectional view schematically showing a constant-temperature apparatus according to embodiment 4;

FIG. 9B schematically shows the flow of a tank gas;

FIG. 10A is a vertical cross-sectional view schematically showing the gas sensor according to variation 3;

FIG. 10B is a vertical cross-sectional view schematically showing the gas sensor according to variation 4;

FIG. 11A is a vertical cross-sectional view schematically showing the gas sensor according to embodiment 5;

FIG. 11B schematically shows the end face of the gas sensor according to embodiment 5 at the second end;

FIG. 12A is a vertical cross-sectional view schematically showing the gas sensor according to variation 5;

FIG. 12B schematically shows the end face of the gas sensor according to variation 5 at the second end;

FIG. 13 is a vertical cross-sectional view schematically showing the gas sensor according to embodiment 6;

FIG. 14A is a vertical cross-sectional view schematically showing the gas sensor according to embodiment 7; and

FIG. 14B is a horizontal cross-sectional view schematically showing the gas sensor according to embodiment 7.

### DETAILED DESCRIPTION OF THE INVENTION

The invention will now be described by reference to the preferred embodiments. This does not intend to limit the scope of the present invention, but to exemplify the invention.

A description will be given of suitable embodiments of the present invention with reference to the drawings. The embodiments do not intend to limit the scope of the invention but exemplify the invention. Not all of the features and the combinations thereof described in the embodiments are necessarily essential to the invention. Like numerals are used to represent like elements, members, and processes and a description will be omitted as appropriate. The scales and shapes shown in the figures are defined for convenience's sake to make the explanation easy and shall not be interpreted limitatively unless otherwise specified. Terms like “first”, “second”, etc. used in the specification and claims do not indicate an order or importance by any means and are used to distinguish a certain feature from the others.

### Embodiment 1

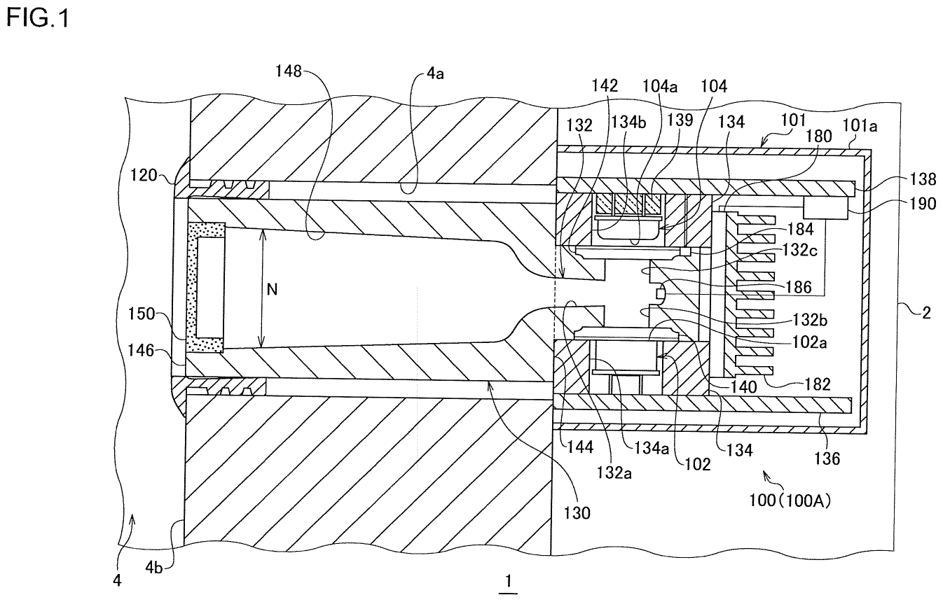

FIG. 1 is a horizontal cross-sectional view showing a part of a constant-temperature apparatus according to embodiment 1. FIG. 1 shows a cross-sectional shape of a gas sensor 100 observed from above in a vertical direction. The constant-temperature apparatus 1 according to this embodiment is provided with a casing 2, a constant-temperature tank 4, and a gas sensor 100 (100A). The constant-temperature apparatus 1 according to this embodiment is exemplified by a CO₂ incubator provided with a dry-heat sterilization function. The casing 2 forms the outer casing of the constant-temperature apparatus 1. The constant-temperature tank 4 is provided inside the casing 2. The constant-temperature tank 4 houses a culture such as cells. The constant-temperature apparatus 1 is configured such that the culture can be transported into or retrieved from the constant-temperature tank 4 via an outer door (not shown) provided in the casing 2 and an inner door (not shown) provided in the constant-temperature tank 4. The constant-temperature tank 4 houses a gas (hereinafter, referred to as a tank gas) containing carbon dioxide (CO₂), etc.

The gas sensor 100 is a sensor for detecting the density and humidity of a predetermined gas (hereinafter, referred to as a gas subject to detection) contained in the tank gas in the constant-temperature tank 4. The gas subject to detection is exemplified by CO₂. The gas sensor 100 transmits a signal indicating the result of detection to the controller (not shown) of the constant-temperature apparatus 1. The controller controls the entirety of the constant-temperature apparatus 1 by managing the temperature or humidity of the constant-temperature tank 4, managing the density of the gas subject to detection, driving a circulation fan, etc. The gas sensor 100 is inserted into and fixed in a through hole 4a that communicates spaces inside and outside the constant-temperature tank 4. An adiabatic member (not shown) is provided in the space between the casing 2 and the constant-temperature tank 4. The gas sensor 100 is provided with a gas detection unit 101 and a gas passage 130.

### [Gas Detection Unit]

The gas detection unit 101 includes a light source 102, a density detector 104, a gas introduction chamber 132, a bracket 134, a first translucent member 140, a second translucent member 142, a temperature adjustment unit 180, and a humidity detection unit 190. These members are housed in a casing 101a.

The light source 102 emits a first light of a predetermined wavelength. The first light is a light of a wavelength absorbed by the gas subject to detection. In the case the gas subject to detection is CO₂, the first light is a light of a wavelength of, for example, 4.64 μm. Further, the light source 102 is preferably an infrared light source. The light source 102 is exemplified by a thermal infrared light source comprised of a black-body coating and emits infrared light over an extensive wavelength range. A thermal infrared light source that emits infrared light from a high-temperature heat generator is a mainstream infrared light source. Examples of the heat generator include a filament, a ceramic, and a coating. An LED may be used in the light source 102. The light source 102 is mounted on a first substrate 136 and is electrically connected to a wiring pattern (not shown) on the first substrate 136. The light source 102 is controlled to be turned on and off by, for example, the controller of the constant-temperature apparatus 1.

The density detector 104 receives the first light emitted from the light source 102 and detects the density of the gas subject to detection based on absorption of first light by the gas subject to detection. More specifically, a light receiving device (not shown) of the density detector 104 receives the first light emitted from the light source 102, and the density detector 104 detects the presence and the density of the gas subject to detection based on the variation in light intensity caused by the absorption of light by the gas subject to detection. The density detector 104 is exemplified by an infrared sensor configured to absorb infrared light and output an electrical signal. The infrared sensor is exemplified by a quantum type sensor such as a photodiode and a photoconductor configured to output a signal by photoelectric conversion, or a thermal type sensor such as a thermopile and a pyroelectric sensor configured to convert temperature variation due to infrared absorption into an electric signal.

The density detector 104 is mounted on a second substrate 138 and is electrically connected to a wiring pattern (not shown) on the second substrate 138. The density detector 104 outputs a signal indicating the density of the gas subject to detection to the controller of the constant-temperature apparatus 1. The density detector 104 may deliver the value indicating the amount of the first light received itself to the controller, as a signal indicating the density of the gas subject to detection. In this case, the controller of the constant-temperature apparatus 1 converts the amount of received light into the density. The density detector 104 also transmits a signal indicating the amount of received light to the humidity detection unit 190. The density detector 104 is easily affected by the ambient temperature. For this reason, an adiabatic member 139 is provided between the density detector 104 and the second substrate 138. The adiabatic member 139 inhibits conduction of heat from the second substrate 138 to the density detector 104.

The light source 102 and the density detector 104 are provided such that a light emitting surface 102a of the light source 102 and a light receiving surface 104a of the density detector 104 face each other. This increases the efficiency of guiding the light from the light source 102 to the density detector 104. The gas introduction chamber 132 through which the gas subject to detection flows in is provided between the light source 102 and the density detector 104. The gas introduction chamber 132 includes a first space 132a, a second space 132b, and a third space 132c. The first space 132a extends in a direction that intersects a direction in which the light source 102 and the density detector 104 are arranged and is connected to the gas passage 130. The second space 132b extends from the first space 132a toward the light source 102. The third space 132c extends from the first space 132a toward the density detector 104.

The tank gas in the constant-temperature tank 4 flows into the first space 132a via the gas passage 130. The tank gas passing through the first space 132a flows into the second space 132b and the third space 132c. By providing the second space 132b between the first space 132a and the light source 102 and providing the third space 132c between the first space 132a and the density detector 104, the flow passage length of the tank gas from the constant-temperature tank 4 to the light source 102/the density detector 104 is extended. This lowers the temperature of the tank gas approaching the light source 102 and the density detector 104 and so inhibits an increase in the temperature of the light source 102 and the density detector 104.

A certain distance (optical distance) needs to be provided between the light source 102 and the density detector 104 for measurement of the gas subject to detection. In a structure where the first space 132a is connected to the light source 102 and the density detector 104 without interposing the second space 132b and the third space 132c, the width of the first space 132a needs to be extended to the optical distance. This might make it difficult to secure the strength of the member defining the gas introduction chamber 132. By providing the second space 132b and the third space 132c, on the other hand, the width of the first space 132a can be smaller than the optical distance. In this way, the strength of the member defining the gas introduction chamber 132 is secured more properly.

Preferably, the wall surface defining the second space 132b and the wall surface defining the third space 132c are coated with a metal film. The metal film may for example be made of a metal having a high reflectance in the infrared range such as gold, aluminum, chrome, etc. By providing a metal film, the light from the light source 102 is inhibited from being absorbed by the wall surface of the second space 132b and the third space 132c and the efficiency of guiding the light from the light source 102 to the density detector 104 is increased. As a result, the sensitivity of detection by the gas sensor 100 is increased.

The light source 102 and the density detector 104 are supported by a bracket 134. The bracket 134 is made of a material having a high thermal conductivity such as aluminum. The bracket 134 includes a first housing 134a and a second housing 134b.

The first housing 134a is provided adjacent to the second space 132b of the gas introduction chamber 132. The first housing 134a houses the light source 102. The light source 102 is provided such that the light emitting surface 102a faces the second space 132b. The first housing 134a has an opening toward the second space 132b. The opening is blocked by the first translucent member 140. Accordingly, the gas introduction chamber 132 and the first housing 134a are spaced apart from each other by the first translucent member 140, and the tank gas is inhibited from leaking into the first housing 134a. This inhibits the accuracy of detection by the gas sensor 100 from being lowered. In other words, the first translucent member 140 functions as a lid member. Preferably, the opening in the first housing 134a is hermetically sealed by the first translucent member 140. The first translucent member 140 is in contact with the bracket 134.

The second housing 134b is a space provided adjacent to the third space 132c of the gas introduction chamber 132. The second housing 134b houses the density detector 104. The density detector 104 is provided such that the light receiving surface 104a faces the third space 132c. The second housing 134b has an opening toward the third space 132c. The opening is blocked by the second translucent member 142. Accordingly, the gas introduction chamber 132 and the second housing 134b are spaced apart from each other by the second translucent member 142, and the tank gas is inhibited from leaking into the second housing 134b. This inhibits the accuracy of detection by the gas sensor 100 from being lowered. In other words, the second translucent member 142 functions as a lid member. Preferably, the opening in the second housing 134b is hermetically sealed by the second translucent member 142. The second translucent member 142 is in contact with the bracket 134.

The first translucent member 140 and the second translucent member 142 provided between the light source 102 and the density detector 104 are made of a material that transmits the light from the light source 102 (i.e., the ratio of absorption of emitted light is low). Therefore, the first translucent member 140 and the second translucent member 142 form an optical window. In this embodiment, infrared light is emitted from the light source 102. Therefore, the first translucent member 140 and the second translucent member 142 are made of, for example, germanium, silicon, sapphire, etc.

The density detector 104 and the second translucent member 142 are spaced apart from each other. By providing a space between the density detector 104 and the second translucent member 142, heat conduction from the second translucent member 142 to the density detector 104 is inhibited. This improves the accuracy of detection by the gas sensor 100. Further, the density detector 104 is provided to be spaced apart from the wall surface of the second housing 134b. This inhibits the heat from being conducted to the density detector 104 via the bracket 134.

Meanwhile, the light source 102 and the first translucent member 140 may or may not be in contact with each other. In the case the light source 102 and the first translucent member 140 are in contact with each other, the heat of the light source 102 can be conducted efficiently by the first translucent member 140. This inhibits dew condensation on the first translucent member 140 from occurring when the density of the gas subject to detection is measured. By inhibiting dew condensation, the efficiency of light guidance from the light source 102 to the density detector 104 is inhibited from being lowered.

The temperature adjustment unit 180 varies the temperature of the first translucent member 140 and the second translucent member 142. By way of one example, the temperature adjustment unit 180 is comprised of a temperature variable device such as a Peltier device. One surface of the temperature adjustment unit 180 is in contact with the bracket 134 in a manner that heat can be conducted. A heat dissipating fin 182 is in contact with the other surface of the temperature adjustment unit 180 in a manner that heat can be conducted. By way of one example, the driving of the temperature adjustment unit 180 is controlled by the humidity detection unit 190. The temperature adjustment unit 180 is capable of giving heat to or depriving heat from the first translucent member 140 and the second translucent member 142 via the bracket 134. The heat of the temperature adjustment unit 180 is dissipated outside via the heat dissipating fin 182.

The gas detection unit 101 includes a first temperature sensor 184 for sensing the temperature of the second translucent member 142. A publicly known sensor such as a thermocouple and a thermistor can be used for the first temperature sensor 184. By way of one example, the first temperature sensor 184 is electrically connected to the second substrate 138. The first temperature sensor 184 outputs a signal indicating the temperature of the second translucent member 142 to the humidity detection unit 190 via the second substrate 138. In the case the temperature adjustment unit 180 is provided with a feature capable of sensing the temperature of the second translucent member 142, the temperature adjustment unit 180 can also function as the first temperature sensor 184.

Further, the gas detection unit 101 includes a second temperature sensor 186 for sensing the ambient temperature of the gas sensor 100. A publicly known sensor such as a thermocouple and a thermistor can be used for the second temperature sensor 186. By way of one example, the second temperature sensor 186 is provided in the gas introduction chamber 132. The second temperature sensor 186 outputs a signal indicating the ambient temperature of the gas sensor 100 to the humidity detection unit 190. The temperature sensor provided in the constant-temperature apparatus 1 to sense the temperature in the constant-temperature tank 4 can be used as the second temperature sensor 186.

The humidity detection unit 190 detects the humidity of the gas subject to detection based on the variation in the amount of the first light received in the density detector 104 and by using the temperature of the second translucent member 142 and the ambient temperature of the gas sensor 100. The humidity detection unit 190 is implemented in hardware by a device or a circuit such as a CPU and a memory of a computer, and in software by a computer program, etc. FIG. 1 depicts the humidity detection unit 190 as a functional block. It will be understood by those skilled in the art that the functional block may be implemented in a variety of manners by a combination of hardware and software. The humidity detection unit 190 outputs a signal indicating the humidity of the gas subject to detection to the controller of the constant-temperature apparatus 1. In this embodiment, the humidity detection unit 190 is provided in the casing 101a, but the configuration is not limited to that of the embodiment. For example, the controller of the constant-temperature apparatus 1 may function as the humidity detection unit 190.

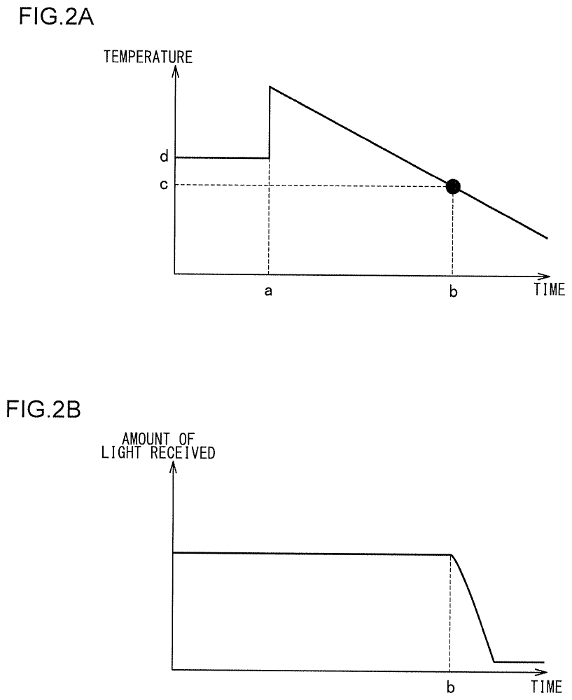

A description will now be given of a principle of detecting the humidity of the gas subject to detection. FIG. 2A is a graph showing a temperature variation in the first translucent member and the second translucent member that occurs when the humidity is detected. FIG. 2B is a graph showing a variation in the amount of light received by the density detector when the humidity is detected. Humidity detection is started by first increasing the temperature of the first translucent member 140 and the second translucent member 142 (time a in FIG. 2A). Subsequently, the temperature of the first translucent member 140 and the second translucent member 142 is gradually lowered.

As the temperature of the first translucent member 140 and the second translucent member 142 is lowered, dew condensation occurs in the first translucent member 140 and the second translucent member 142 at a certain point of time. When dew condensation occurs, the first light emitted from the light source 102 is diffused in part as it passes through the first translucent member 140 and the second translucent member 142. This decreases the amount of light received by the density detector 104. Therefore, the temperature of the first translucent member 140 and the second translucent member 142 occurring at the point of time (time b in FIG. 2A and FIG. 2B) when the amount of light received by the density detector 104 begins to be decreased will be the dew-point temperature c. When the dew-point temperature c and the ambient temperature of the gas sensor 100 are known, the absolute humidity of the gas subject to detection is calculated according to a publicly known calculating formula.

In this embodiment, the temperature of the second translucent member 142 is sensed by the first temperature sensor 184 and sent to the humidity detection unit 190. Therefore, the temperature of the second translucent member 142 occurring when the amount of light received begins to be attenuated is used as the dew-point temperature c. Further, the ambient temperature of the gas sensor 100 is sensed by the second temperature sensor 186 and sent to the humidity detection unit 190. Further, the signal indicating the amount of light received is transmitted from the density detector 104 to the humidity detection unit 190 at predetermined intervals. Therefore, the humidity detection unit 190 can know the point of time when dew condensation occurs, based on the signal received from the density detector 104. The humidity detection unit 190 can determine the signal from the first temperature sensor 184 received when dew condensation occurs to be the signal indicating the ambient temperature c. The humidity can be equally calculated by using the temperature of the first translucent member 140.

The light source 102 and the density detector 104 communicate with a space outside the gas sensor 100, and, ultimately, the space outside the constant-temperature apparatus 1. More specifically, the first housing 134a also has an opening on the side opposite to the second space 132b. Similarly, the second housing 134b also has an opening on the side opposite to the third space 132c. For example, the first housing 134a and the second housing 134b are comprised of a through hole provided in the bracket 134. Further, an opening (not shown) is provided in the casing 101a. This allows the light source 102 and the density detector 104 to communicate with the space outside the gas sensor 100 and a space outside the constant-temperature apparatus 1.

With such a configuration, air can be ventilated between the area where the light source 102 and the density detector 104 are located and the space outside. The ventilation inhibits the accuracy of detection by the gas sensor 100 from being lowered due to the leakage of the tank gas from a gap between a packing 120, described later, and the through hole 4a, a gap between the packing 120 and the gas sensor 100, or a gap between the bracket 134 and the first translucent member 140 or the second translucent member 142, etc.

### [Gas Passage]

The gas passage 130 is a passage for the tank gas containing the gas subject to detection and is interposed between the constant-temperature tank 4 and the gas detection unit 101. By providing the gas passage 130, the gas detection unit 101 is spaced apart from the constant-temperature tank 4. This inhibits conduction of heat from an internal space of the constant-temperature tank 4 to the light source 102 and the density detector 104. As a result, damage to the light source 102 and the density detector 104 from the heat is inhibited so that the accuracy of detection by the gas sensor 100 is inhibited from being lowered.

The gas passage 130 is comprised of a tubular member and includes a first end 144, a second end 146 opposite to the first end 144, and a hollow part 148 extending from the first end 144 to the second end 146. The first end 144 is provided toward the gas detection unit 101. In other words, the light source 102, the density detector 104, and the two translucent members are provided on the side of the first end 144. The second end 146 is provided toward the gas space where the gas subject to detection is located, i.e., toward the constant-temperature tank 4.

The end of hollow part 148 toward the first end 144 is connected to the first space 132a of the gas introduction chamber 132. The end of the hollow part 148 toward the second end 146 is connected to the internal space of the constant-temperature tank 4. A cap 150 is laid in the end of the hollow part 148 toward the second end 146. The cap 150 is made of a porous material having heat resistance and water repellency. For example, the cap 150 is made of a resin material like polytetrafluoroethylene (PTFE), a metal mesh such as SUS, a punching metal, an expanded metal, etc. The heat resistance of the cap 150 is preferably 200° or higher. The cap 150 allows passage of the tank gas.

The tank gas containing the gas subject to detection is circulated between the first end 144 and the second end 146, i.e., between the constant-temperature tank 4 and the gas detection unit 101 via the hollow part 148. The hollow part 148 has, at least in part, a shape in which the cross-sectional area N of the flow passage grows smaller away from the second end 146 and toward the first end 144 either in steps or continuously. In other words, the hollow part 148 is shaped such that the area of the cross section perpendicular to the direction in which the first end 144 and the second end 146 are arranged grows smaller away from the second end 146 and toward the first end 144 either in steps or continuously. The hollow part 148 shown in FIG. 1 is shaped such that the cross-sectional area N of the flow passage grows smaller continuously from the second end 146 toward the first end 144.

A large cross-sectional area N of the flow passage toward the second end 146 makes it easier to introduce the tank gas into the hollow part 148. Meanwhile, a small cross-sectional area N toward the first end 144 reduces the distance between the light source 102 and the density detector 104. The distance between the light source 102 and the density detector 104 in this embodiment is, for example, about 10 mm. By reducing the distance between the light source 102 and the density detector 104, the light intensity of the light source 102 necessary for detection of the gas subject to detection is reduced. In other words, the gas subject to detection can be measured with a lower power. Another advantage is that the size of the gas detection unit 101 is prevented from growing.

The gas passage 130 is made of an adiabatic material at least in part. The gas passage 130 according to this embodiment is made of an adiabatic material in its entirety. Where only a part of the gas passage 130 is made of an adiabatic material, it is preferable to provide the adiabatic material such that the non-adiabatic material is discontinuous at some position between the first end 144 and the second end 146. By configuring at least a part of the gas passage 130 to be made of an adiabatic material, the heat in the internal space of the constant-temperature tank 4 is inhibited from being conducted to the light source 102 and the density detector 104 via the gas passage 130.

For example, an adiabatic material that causes the neighborhood of the light source 102 and the density detector 104 to be at a temperature of 100° C. or lower when the temperature inside the constant-temperature tank 4 is 190° C. is selected. By selecting an adiabatic material having a high heat resistance, the adiabatic effect is enhanced. A high-temperature resistant resin is suitable as the adiabatic material. This is because a high-temperature resistant resin can be worked more easily and is more heat resistant than a metal. Specific examples of the adiabatic material include: polyphenylene sulfide (PPS); a fluororesin like polytetrafluoroethylene (PTFE) and Teflon (registered trademark); polyether ether ketone (PEEK); silicon resin; and polyamide-imide (PAI).

In this embodiment, the gas passage 130 and the gas introduction chamber 132 of the gas detection unit 101 have an integrally molded structure made of an adiabatic material. In other words, the hollow part 148, the first space 132a, the second space 132b, and the third space 132c are defined inside a one-piece tubular member made of an adiabatic material.

The gas sensor 100 is fixed relative to the constant-temperature tank 4 by laying the packing 120 between the outer side surface of the gas passage 130 and the inner side surface of the through hole 4a while the gas passage 130 is being inserted into the through hole 4a of the constant-temperature tank 4. For example, the packing 120 is made of a silicon resin. The gas sensor 100 is configured such that the second end 146 of the gas passage 130 is exposed in the constant-temperature tank 4, the gas passage 130 is located in the through hole 4a, and the gas detection unit 101 is located outside the constant-temperature tank 4.

The gas sensor 100 is provided such that the gas passage 130 extends horizontally while the gas sensor 100 is being fixed relative to the constant-temperature tank 4. In other words, the gas sensor 100 is provided such that the first end 144 and the second end 146 are arranged in the horizontal direction. Thus, by placing the gas sensor 100 in the horizontal arrangement in this way, the dust or liquid is inhibited from entering the gas passage 130 or the gas introduction chamber 132, and the dust or liquid that has entered is inhibited from being collected inside. This inhibits the accuracy of detection by the gas sensor 100 from being lowered.

The hollow part 148 and the first space 132a extend horizontally and in a direction normal to a wall surface 4b of the constant-temperature tank 4 while the gas sensor 100 is being fixed to the constant-temperature tank 4. The second space 132b and the third space 132c extend horizontally and parallel to the wall surface 4b of the constant-temperature tank 4. The light source 102 and the density detector 104 are arranged in the horizontal direction. By providing the light source 102 and the density detector 104 on the side surfaces of the gas sensor 100, the dust or moisture is inhibited from being collected on the light source 102 or the density detector 104.

Further, the hollow part 148 has a portion substantially shaped in a truncated cone having a bottom surface located toward the second end 146 and a top surface located toward the first end 144. Therefore, the lower surface of the hollow part 148 in the vertical direction is tapered at least in part so as to incline downward in the vertical direction away from the first end 144 and toward the second end 146. This makes it easy for the dust or liquid to be discharged from inside the hollow part 148 or the gas introduction chamber 132 so that the dust or liquid is more properly inhibited from being collected. The hollow part 148 shown in FIG. 1 is shaped such that the lower surface thereof in the vertical direction inclines continuously from the second end 146 toward the first end 144.

The tank gas in the constant-temperature tank 4 flows into the hollow part 148 via the cap 150. The tank gas flowing into the hollow part 148 advances toward the first end 144 and flows into the first space 132a of the gas introduction chamber 132. The tank gas flowing into the first space 132a flows into the second space 132b and the third space 132c. As a result, the first space 132a-the third space 132c are filled with the tank gas.

A description will now be given of the operation of the gas sensor 100. In a situation in which the temperature of the first translucent member 140 and the second translucent member 142 is the first temperature d (see FIG. 2A) higher than the dew-point temperature c of the gas subject to detection, the density detector 104 detects the density of the gas subject to detection. For example, the heat of the light source 102 and the heat of the tank gas filling the gas introduction chamber 132 maintain the temperature of the first translucent member 140 and the second translucent member 142 at the first temperature d without relying on the temperature adjustment unit 180. More preferably, the temperature of the first translucent member 140 and the second translucent member 142 is maintained at a temperature higher than the ambient temperature. The temperature adjustment unit 180 may heat the first translucent member 140 and the second translucent member 142. This inhibits occurrence of dew condensation in the first translucent member 140 and the second translucent member 142 more properly.

More specifically, the light of the light source 102 including the first light is emitted toward the gas subject to detection. In other words, the light of the light source 102 is emitted toward the second space 132b filled with the tank gas that contains the gas subject to detection. The light emitted from the light source 102 arrives at the density detector 104 via the first translucent member 140, the second space 132b, the area in the first space 132a sandwiched by the second space 132b and the third space 132c, the third space 132c, and the second translucent member 142. In this process, the first light of a predetermined wavelength is absorbed by the gas subject to detection located in the first space 132a-the third space 132c. The density detector 104 can detect the presence and density of the gas subject to detection based on the variation in the amount of the first light.

In this embodiment, the light source 102 emits infrared light, and the first light of a wavelength 4.26 μm is absorbed by CO₂ located in the first space 132a-the third space 132c. The density detector 104 is capable of detecting the presence and density of CO₂ based on the intensity (amount of light) of the first light received by the light receiving device, with reference to the intensity (amount of light) of the first light in the light emitted from the light source 102. For example, the density of CO₂ that the gas sensor 100 is capable of detecting is 0-20%.

The humidity detection unit 190 drives the temperature adjustment unit 180 according to a predetermined timing schedule. The temperature adjustment unit 180 absorbs the heat from the first translucent member 140 and the second translucent member 142 to lower the temperature of the respective translucent members gradually. Preferably, the temperature adjustment unit 180 temporarily raises the temperature from the first temperature d and then gradually lowers the temperature, as shown in FIG. 2A. When the temperature of the first translucent member 140 and the second translucent member 142 reaches the dew-point temperature c, the moisture in the gas subject to detection is condensed on the surface of the first translucent member 140 and the second translucent member 142.

This will cause a predetermined decrease in the amount of the first light received by the density detector 104. The humidity detection unit 190 detects the humidity of the gas subject to detection, based on the point of time when the decrease in the amount of the first light received is sensed. In other words, when the humidity detection unit 190 senses a decrease in the amount of the first light received, the humidity detection unit 190 calculates the absolute humidity of the gas subject to detection by using the result of sensing by the first temperature sensor 184, i.e., the dew-point temperature c, and the result of sensing by the second temperature sensor 186, i.e., the ambient temperature, which are received concurrently with the decrease. The “predetermined decrease” sensed by the humidity detection unit 190 can be defined as appropriate based on experiments or simulation by the designer.

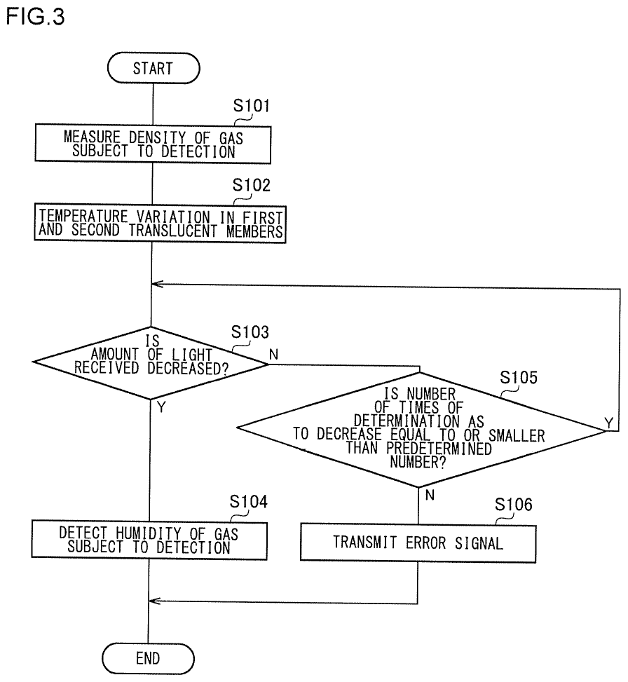

As described above, the gas sensor 100 performs density detection and humidity detection of the gas subject to detection in a time-divided manner by varying the temperature of the first translucent member 140 and the second translucent member 142. Density detection and humidity detection may be alternately repeated at a predetermined period or may be performed when an instruction to perform density detection or humidity detection transmitted from the controller of the constant-temperature apparatus 1 is received by the gas sensor 100. By way of one example, the operation flow of the gas sensor 100 performed when density detection and humidity detection are alternately repeated will be described. FIG. 3 is a flowchart showing the operation of the gas sensor according to embodiment 1. The flow is executed repeatedly according to a predetermined timing schedule in a situation in which the constant-temperature apparatus 1 is in operation and the light source 102 is lighted.

First, the density detector 104 detects the density of the gas subject to detection based on the amount of the first light received (S101). The temperature adjustment unit 180 then temporarily raises the temperature of the first translucent member 140 and the second translucent member 142 and then gradually lowers the temperature (S102). The humidity detection unit 190 determines whether the amount of the first light received in the density detector 104 is decreased (S103). When the amount of the first light received is decreased (Y in S103), the humidity detection unit 190 calculates the absolute humidity of the gas subject to detection by using the result of sensing by the first temperature sensor 184 and the result of sensing by the second temperature sensor 186, which are received concurrently with the decrease in the amount of light received (S104).

When the amount of the first light received is not decreased (N in S103), the humidity detection unit 190 determines whether the number of times of determination as to a decrease in the amount of the first light received is equal to or smaller than a predetermined number (S105). When the number of times of determination is equal to or smaller than the predetermined number (Y in S105), the humidity detection unit 190 determines whether the amount of the first light received is decreased again (S103). When the number of times of determination exceeds the predetermined number (N in S105), the humidity detection unit 190 transmits an error signal to the controller of the constant-temperature apparatus 1 (S106). In this flow, a determination on an error is made in step S105 based on the number of times of determination as to a decrease in the amount of light received. Alternatively, a determination on an error may be made based on, for example, the time elapsed since the temperature is started to be changed by the temperature adjustment unit 180. The “predetermined number” can be defined as appropriate based on experiments or simulation by the designer.

As described above, the gas sensor 100 according to this embodiment is provided with the light source 102 for emitting the first light toward the gas subject to detection, and the density detector 104 that receives the first light and detects the density of the gas subject to detection based on absorption of the first light. The gas sensor 100 is also provided with the first translucent member 140 and the second translucent member 142 provided between the light source 102 and the density detector 104, the temperature adjustment unit 180 for varying the temperature of the translucent members, and the humidity detection unit 190. The humidity detection unit 190 detects the humidity of the gas subject to detection based on the variation in the amount of the first light received in the density detector 104 and by using the temperature of the second translucent member 142 and the ambient temperature of the gas sensor 100.

In a situation in which the temperature of the first translucent member 140 and the second translucent member 142 is the first temperature d higher than the dew-point temperature of the gas subject to detection, the gas sensor 100 detects the density of the gas subject to detection using the density detector 104. Further, the temperature adjustment unit 180 gradually lowers the temperature of the first translucent member 140 and the second translucent member 142, and the humidity detection unit 190 detects the humidity of the gas subject to detection based on the point of time when a predetermined decrease in the amount of the first light received in the density detector 104 occurs.

Thus, according to the gas sensor 100 of the embodiment, the density and humidity of the gas subject to detection can be detected by using a single sensor. Further, the optical system (the light source, the density detector, and the translucent members) used to detect the density of the gas subject to detection are also used in detecting the humidity of the gas subject to detection. In other words, a single optical system is capable of detecting the density and humidity of the gas subject to detection. Accordingly, as compared with the case of combining a related-art gas density sensor only capable of detecting the density of the gas subject to detection and a related-art gas humidity sensor only capable of detecting the humidity of the gas subject to detection, the density and humidity of the gas subject to detection can be detected in a simplified configuration. This helps reduce the size and price of the constant-temperature apparatus 1.

In this embodiment, the temperature of both the first translucent member 140 and the second translucent member 142 is varied to induce dew condensation on both of the members. This decreases the amount of the first light received in the density detector 104 more than in the case in which dew condensation is induced only on one of the translucent members. This increases the sensitivity of detection and speed of detection of humidity in the gas sensor 100.

Further, the gas sensor 100 is provided with the gas passage 130. The gas sensor according to the related art is structured such that the light source and the density detector are provided in the constant-temperature tank 4 and are exposed to the gas subject to detection. Meanwhile, the temperature that the light source and the density detector used in a gas sensor can withstand is generally about 100° C. For this reason, the temperature of the gas subject to detection may exceed the withstand temperature of the light source and the density detector and lower the accuracy of detection by the gas sensor. Further, where the related-art gas sensor is mounted in a constant-temperature apparatus such as an incubator, the temperature of the constant-temperature tank may exceed the withstand temperature of the light source and the density detector when the constant-temperature apparatus is sterilized by dry sterilization. In this case, the light source and the density detector will be exposed to a high temperature and the accuracy of detection by the gas sensor may be lowered.

By providing the gas passage 130, the light source 102/the density detector 104 and the constant-temperature tank 4 are thermally isolated. This inhibits damage to the light source 102 and the density detector 104 due to the heat even when the temperature in the constant-temperature tank 4 becomes high such as when the temperature of the tank gas exceeds the withstand temperature of the light source 102 and the density detector 104 or when the constant-temperature tank 4 is sterilized by dry sterilization. Accordingly, the accuracy of detection by the gas sensor 100 is inhibited from belong lowered.

By mounting the gas sensor 100 in the constant-temperature apparatus 1, the density and humidity of the gas subject to detection contained in the tank gas are detected with a high accuracy so that the performance of the constant-temperature apparatus 1 is improved. Since a dry sterilization process can be performed without removing the gas sensor 100, the ease of use of the constant-temperature apparatus 1 is also improved.

In this embodiment, the light source 102 and the density detector 104 are arranged in the horizontal direction, but the embodiment is not limited to this configuration. For example, the light source 102 and the density detector 104 may be arranged in the vertical direction. In this case, it is preferable to provide the light source 102 below. This makes it easy to conduct the heat of the light source 102 to the first translucent member 140. As a result, unintended dew condensation on the first translucent member 140 is inhibited. The following variations of the gas sensor 100 according to embodiment 1 are possible.

### (Variation 1)

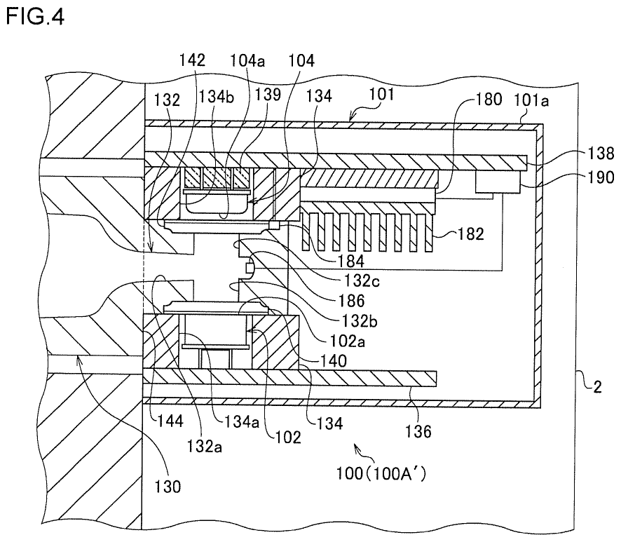

FIG. 4 is a horizontal cross-sectional view schematically showing a part of the gas sensor according to variation 1. Those features of the gas sensor according to this variation that are different from those of embodiment 1 will mainly be described. Common features will be described briefly, or a description thereof will be omitted. The gas sensor 100 (100A′) according to this variation differs from embodiment 1 in that the temperature adjustment unit 180 varies the temperature of only one of the first translucent member 140 and the second translucent member 142. FIG. 4 discloses a structure in which the temperature of the second translucent member 142 is varied by way of example. By configuring only one of the translucent members to be subject to temperature control by the temperature adjustment unit 180, the load imposed on the temperature adjustment unit 180 is reduced. This allows the temperature of the translucent member to be varied more promptly.

In the case only of the translucent members is subject to temperature control by the temperature adjustment unit 180, it is preferred to subject the second translucent member 142 toward the density detector 104 to control. The light source 102 is a heat generating source and generates heat of about 1 W. For this reason, it is more difficult to cool the first translucent member 140 toward the light source 102 than the second translucent member 142. By subjecting the second translucent member 142 to temperature control, therefore, power consumption in the gas sensor 100 is reduced. Further, the temperature of the translucent member is varied more promptly so that the speed of detection of humidity is improved.

### (Variation 2)

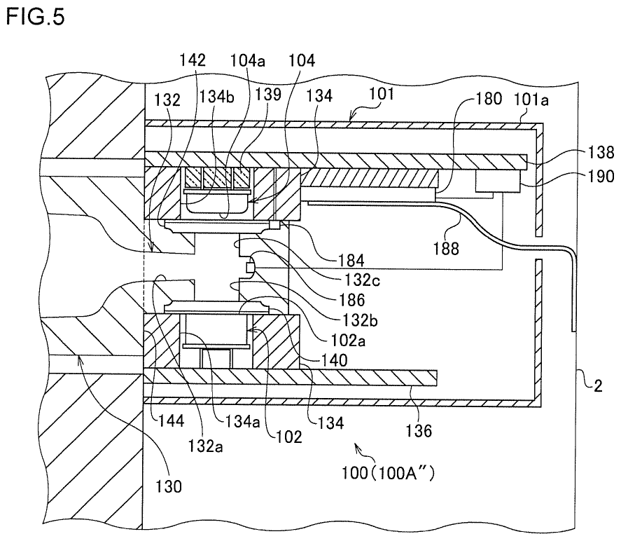

FIG. 5 is a horizontal cross-sectional view schematically showing a part of the gas sensor according to variation 2. Those features of the gas sensor according to this variation that are different from those of variation 1 will mainly be described. Common features will be described briefly, or a description thereof will be omitted. The structure of the gas sensor 100 (100A″) according to this variation for dissipating the heat of the temperature adjustment unit 180 differs from that of variation 1.

More specifically, the gas sensor 100 according to this variation is configured such that the temperature adjustment unit 180 varies the temperature of only one of the first translucent member 140 and the second translucent member 142. FIG. 5 discloses a structure in which the temperature of the second translucent member 142 is varied by way of example. This allows the temperature of the translucent member to be varied more promptly.

In the gas sensor 100 according to this variation, a heat conducting sheet 188 is used in place of the heat dissipating fin 182. One end of the heat conducting sheet 188 is in contact with the temperature adjustment unit 180. The other end of the heat conducting sheet 188 is in contact with, for example, the casing 2. This dissipates the heat of the temperature adjustment unit 180 to the casing 2 via the heat conducting sheet 188. By using the heat conducting sheet 188, the heat dissipating fin 182 can be omitted so that the cost of the gas sensor 100 is reduced. Further, the efficiency of dissipating the heat of the temperature adjustment unit 180 is improved. Any of various structures capable of conducting heat such as a metallic sheet, a graphite sheet, and a heat pipe can be used as the heat conducting sheet 188.

### Embodiment 2

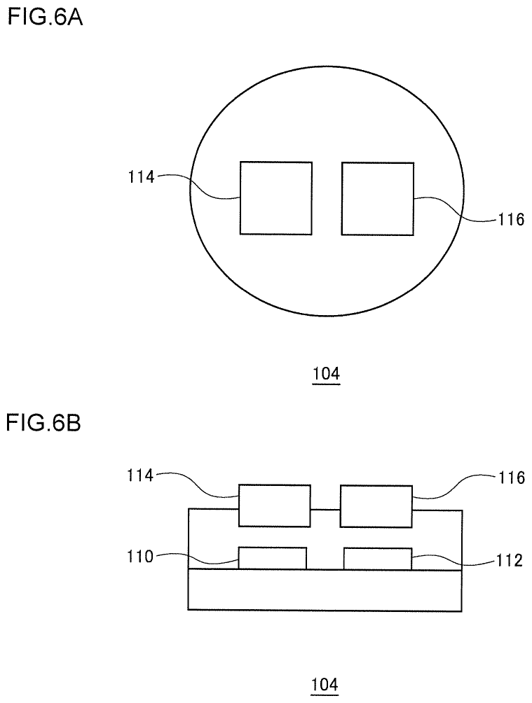

FIG. 6A is a plan view schematically showing the density detector provided in the gas sensor according to embodiment 2. FIG. 6B is a side view schematically showing the density detector provided in the gas sensor according to embodiment 2. Those features of the gas sensor according to this embodiment that are different from those of embodiment 1 will mainly be described. Common features will be described briefly, or a description thereof will be omitted. The gas sensor according to this embodiment differs from that of embodiment 1 in that the light source 102 further emits second light, and the density detector 104 receives the second light.

More specifically, the light source 102 (see FIG. 1) of the gas sensor according to this embodiment emits the second light of a wavelength absorbed by water in addition to the first light. The amount of the second light absorbed by water is larger than that of the first light. For example, the wavelength of the second light is 3 μm. Preferably, the light emitted by the light source 102 is infrared light having a wavelength range of 2.7 μm-3.5 μm.

Further, the density detector 104 includes a first detection unit 110, a second detection unit 112, a first optical filter 114, and a second optical filter 116. The first detection unit 110 and the second detection unit 112 are light receiving devices. The first optical filter 114 is provided between the first detection unit 110 and the light source 102 and selectively transmits the first light. The second optical filter 116 is provided between the second detection unit 112 and the light source 102 and selectively transmits the second light. The optical filters are provided directly on or spaced apart from the light receiving surface of the respective detection units.

The density detector 104 selectively detects the first light having a wavelength absorbed by the gas subject to detection by means of the first detection unit 110 and the first optical filter 114. This increases the sensitivity of detection by the gas sensor 100. The first optical filter 114 may be provided in the density detector 104 of embodiment 1. This also provides the same advantage as described above.

In this embodiment, the light source 102 emits the second light having a wavelength absorbed by water. The density detector 104 selectively detects the second light by means of the second detection unit 112 and the second optical filter 116. The humidity detection unit 190 identifies the occurrence of dew condensation based on the amount of the second light received. The second light is more easily absorbed by water than by the gas subject to detection. For this reason, the amount of the second light received by the second detection unit 112 is decreased because the water produced by dew condensation absorbs the second light as well as because the water scatters the second light. In other words, the amount of the second light received is more easily decreased by dew condensation than that of the first light. Accordingly, the sensitivity of detection of humidity by the gas sensor 100 is further increased. Further, the dew-point temperature c is detected more precisely.

According to this embodiment, the density detector 104 detects the density of the gas subject to detection based on the amount of the first light received in a situation in which the temperature of the first translucent member 140 and the second translucent member 142 is the first temperature d higher than the dew-point temperature c of the gas subject to detection. Further, the temperature adjustment unit 180 gradually lowers the temperature of the first translucent member 140 and the second translucent member 142 according to a predetermined timing schedule. When the temperature of the first translucent member 140 and the second translucent member 142 reaches the dew-point temperature c, the moisture in the gas subject to detection is condensed on the surface of the first translucent member 140 and the second translucent member 142.

This will cause a predetermined decrease in the amount of the second light received by the density detector 104. The humidity detection unit 190 detects the humidity of the gas subject to detection, based on the point of time when the decrease in the amount of the second light received is sensed. In other words, when the humidity detection unit 190 senses a decrease in the amount of the second light received, the humidity detection unit 190 calculates the absolute humidity of the gas subject to detection by using the result of sensing by the first temperature sensor 184 and the result of sensing by the second temperature sensor 186, which are received concurrently with the decrease.

### Embodiment 3

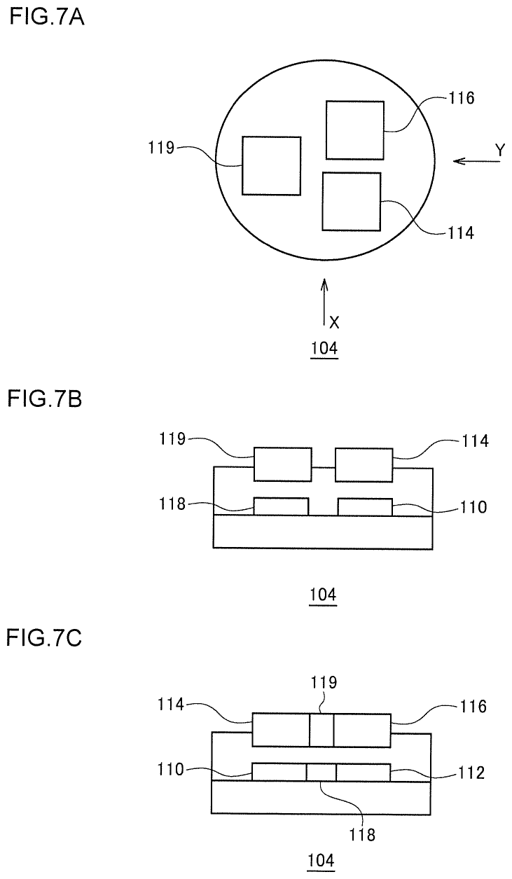

FIG. 7A is a plan view schematically showing the density detector provided in the gas sensor according to embodiment 3. FIGS. 7B and 7C are side views schematically showing the density detector provided in the gas sensor according to embodiment 3. FIG. 7B is a side view in a direction of an arrow X in FIG. 7A, and FIG. 7C is a side view in a direction of an arrow Y in FIG. 7A. Those features of the gas sensor according to this embodiment that are different from those of embodiments 1 and 2 will mainly be described. Common features will be described briefly, or a description thereof will be omitted. The gas sensor according to this embodiment differs from that of embodiment 2 in that the light source 102 further emits third light, and the density detector 104 receives the third light.

More specifically, the light source 102 (see FIG. 1) of the gas sensor emits the third light of a wavelength not absorbed by the gas subject to detection or water in addition to the first light and the second light. For example, the wavelength of the third light is 3.91 μm. “Not absorbed by the gas subject to detection or water” means that the amount of absorption by the gas subject to detection or water is 10% or less, respectively. The third light is used as reference light. In addition to the first detection unit 110, the second detection unit 112, the first optical filter 114, and the second optical filter 116, the density detector 104 further includes a third detection unit 118 and a third optical filter 119. The third detection unit 118 is a light receiving device. The third optical filter 119 is provided between the third detection unit 118 and the light source 102 and selectively transmits the third light. The optical filters are provided directly on or spaced apart from the light receiving surface of the respective detection units.

The density detector 104 selectively detects the first light having a wavelength absorbed by the gas subject to detection by means of the first detection unit 110 and the first optical filter 114. Further, the density detector 104 selectively detect the third light by means of the third detection unit 118 and the third optical filter 119. The density detector 104 detects the density of the gas subject to detection based on the variation in the intensity of the first light and the variation in the intensity of the third light. In other words, the density detector 104 subtracts the amount of decrease in the intensity of the third light from the amount of decrease in the intensity of the first light and defines the difference as the amount of decrease in the intensity of the first light caused by absorption by the gas subject to detection. The density detector 104 detects the density of the gas subject to detection based on the difference. The density detector 104 may send the values indicating the amounts of the first light and the third light received to the controller as signals indicating the density of the gas subject to detection. In this case, the difference between the amounts of the first light and the third light received is converted by the controller of the constant-temperature apparatus 1 into the density of the gas subject to detection.

The third light is not absorbed by the gas subject to detection or water. For this reason, the decrease in the intensity of the third light is caused by external disturbance other than absorption of light by the gas subject to detection or water. The external disturbance includes scattering by the water produced by dew condensation in the first translucent member 140 and the second translucent member 142. Therefore, the density of the gas subject to detection is detected with the decrease in the intensity of the first light caused by external disturbance being excluded, by deriving a difference between the amount of decrease in the intensity of the first light and the amount of decrease in the intensity of the third light. This increases the accuracy of detection by the gas sensor 100. It also allows the density of the gas subject to detection to be detected even when dew condensation occurs in the first translucent member 140 and the second translucent member 142. In accordance with this embodiment, therefore, density measurement and humidity measurement of the gas subject to detection can be performed simultaneously. For example, the humidity of the gas subject to detection can be measured while the density of the gas subject to detection continues to be measured.

Further, the humidity detection unit 190 identifies the occurrence of dew condensation based on the variation in the amount of the second light received. The intensity of the second light is decreased because the water produced by dew condensation absorbs the second light as well as because the water scatters the second light. Accordingly, the sensitivity of detection of humidity by the gas sensor 100 is further increased. Further, the dew-point temperature c is detected more precisely.

According to this embodiment, the density detector 104 detects the density of the gas subject to detection based on the amount of the first light received and the amount of the third light received in a situation in which the temperature adjustment unit 180 gradually lowers the temperature of the first translucent member 140 and the second translucent member 142. Further, the humidity detection unit 190 detects the humidity of the gas subject to detection based on the point of time when a predetermined decrease in the amount of the second light received in the density detector 104 occurs. In other words, when the humidity detection unit 190 senses a decrease in the amount of the second light received, the humidity detection unit 190 calculates the absolute humidity of the gas subject to detection by using the result of sensing by the first temperature sensor 184 and the result of sensing by the second temperature sensor 186 received concurrently with the decrease.

The humidity detection unit 190 may determine the occurrence of dew condensation based on the difference between the amount of decrease in the intensity of the second light and the amount of decrease in the intensity of the third light. In this case, the occurrence of dew condensation can be determined based on the decrease in the intensity of the second light caused only by absorption of the second light by water.

The occurrence of dew condensation can also be sensed by referring to the variation in the amount of the third light received. Therefore, the humidity detection unit 190 may detect the humidity of the gas subject to detection based on the point of time when a predetermined decrease in the amount of the third light received in the density detector 104 occurs. In this case, emission of the second light from the light source 102 and provision of the second detection unit 112 and the second optical filter 116 in the density detector 104 can be omitted.

### Embodiment 4

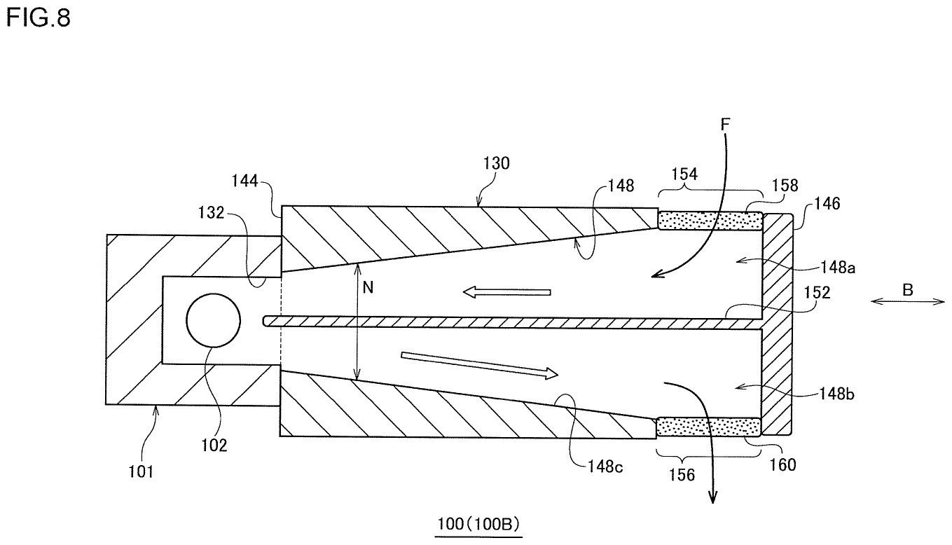

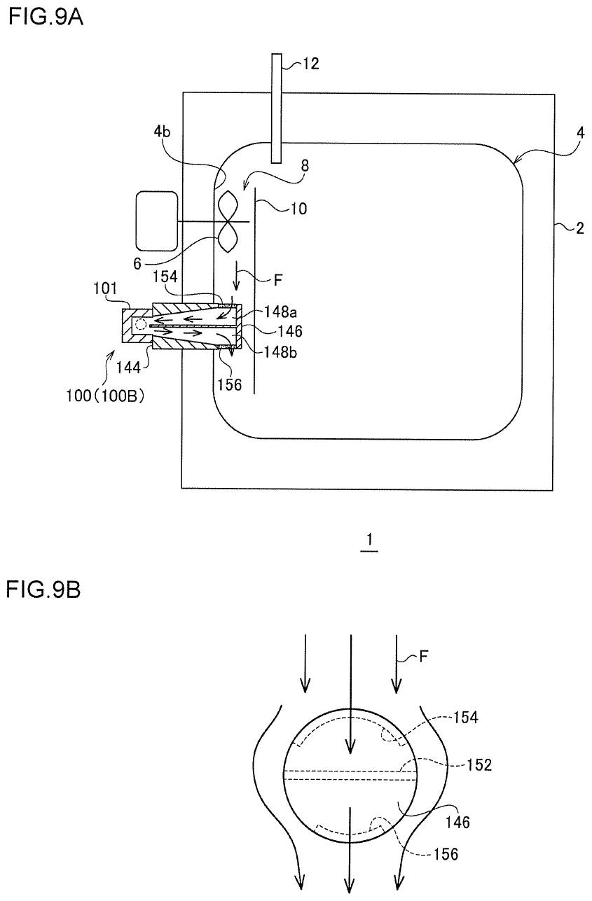

FIG. 8 is a vertical cross-sectional view schematically showing the gas sensor according to embodiment 4. FIG. 9A is a vertical cross-sectional view schematically showing a constant-temperature apparatus according to embodiment 4. FIG. 9B schematically shows the flow of a tank gas. In FIGS. 8 and 9A, illustration of the interior of the gas detection unit 101 is simplified, and illustration of the temperature adjustment unit 180, the heat dissipating fin 182, the humidity detection unit 190, etc. is omitted. Those features of the gas sensor according to this embodiment that are different from those of embodiment 1 will mainly be described. Common features will be described briefly, or a description thereof will be omitted.

The gas passage 130 provided in the gas sensor 100 (100B) according to this embodiment includes a partition member 152. The partition member 152 divides the hollow part 148 into at least two areas including a first area 148a and a second area 148b. In this embodiment, the partition member 152 divides the hollow part 148 into two areas, i.e., the first area 148a and the second area 148b. The partition member 152 extends from the first end 144 to the second end 146. Therefore, each of the first area 148a and the second area 148b extends from the first end 144 to the second end 146. The hollow part 148 is sloped such that a lower surface 148c in the vertical direction descends in the vertical direction away from the first end 144 and toward the second end 146.

Further, the gas passage 130 includes a gas inflow port 154 and a gas outflow port 156. The gas inflow port 154 is provided at the second end 146 and connects the internal space of the constant-temperature tank 4 to the first area 148a. The gas outflow port 156 is provided at the second end 146 and connects the second area 148b to the internal space of the constant-temperature tank 4. The gas inflow port 154 is blocked by a porous member 158, and the gas outflow port 156 is blocked by a porous member 160. The material for forming the porous members 158, 160 is exemplified by the material to form the cap 150. The porous members 158, 160 allow passage of the gas subject to detection.

The aperture planes of gas inflow port 154 and the gas outflow port 156 extend parallel to a direction B (the direction indicated by an arrow B in FIG. 8) in which the first end 144 and the second end 146 are arranged. The fact that the porous member 158 and the porous member 160 extend parallel to the direction B also helps one to understand that the aperture planes of the gas inflow port 154 and the gas outflow port 156 extend parallel to the direction B. In other words, the gas inflow port 154 and the gas outflow port 156 are provided on the side surfaces of the gas passage 130.

The gas sensor 100 is provided on the wall surface 4b of the constant-temperature tank 4 such that the gas inflow port 154 and the gas outflow port 156 project from the wall surface 4b of the constant-temperature tank 4 into the internal space. Normally, a flow of tank gas containing the gas subject to detection (the gas flow F) exits in the constant-temperature tank 4. The gas inflow port 154 is provided such that the aperture plane intersects the gas flow F in the constant-temperature tank 4, i.e., to intersect the direction of flow of the gas subject to detection. Preferably, the gas inflow port 154 is provided such that the aperture plane is perpendicular to the gas flow F. Further, the gas outflow port 156 is provided opposite to the gas inflow port 154 in the direction of the gas flow F.

Further, the gas inflow port 154 is provided upstream in the gas flow F, and the gas outflow port 156 is provided downstream of the gas inflow port 154 in the gas flow F. Further, the second end 146 is provided in an area where the gas subject to detection flows downward in the vertical direction in the constant-temperature tank 4. Further, the gas inflow port 154 is provided above the gas outflow port 156 in the vertical direction.

The tank gas located in the constant-temperature tank 4 flows into the hollow part 148 from the gas inflow port 154, flows in the first area 148a toward the first end 144, and arrives at the gas detection unit 101, and, more specifically, at the space between the light source 102 and the density detector 104. In association with this, the tank gas located in the gas detection unit 101 flows in the second area 148b toward the second end 146 and flows out from the gas outflow port 156 into the constant-temperature tank 4.

Thus, by using the partition member 152 to divide the hollow part 148 into the first area 148a and the second area 148b, providing the gas inflow port 154 in the first area 148a, and providing the gas outflow port 156 in the second area 148b, the flow of the tank gas in the hollow part 148 is straightened to create a convection flow. The feature makes it possible to introduce the gas subject to detection into the gas detection unit 101 more efficiently and, accordingly, to replace the gas in the gas introduction chamber 132 promptly. Further, since a high-speed gas flow can be generated, it is possible to induce dew condensation on and dry the first translucent member 140 and the second translucent member 142 promptly. Accordingly, the speed of detection and the sensitivity of detection by the gas sensor 100 are improved.

By projecting the second end 146 of the gas sensor 100 into the area where the gas flow F is located, the pressure applied on the surface upstream in the gas flow F, i.e., the pressure applied to the surface directly hit by the gas flow F, will be higher than the pressure applied to the downstream surface, i.e., the pressure applied to the surface reached by the gas flow F in a roundabout fashion. Therefore, a pressure difference is created between the upstream surface and the downstream surface. Thus, by providing the gas inflow port 154 on the upstream side in the gas flow F and providing the gas outflow port 156 on the downstream side, the pressure difference can be utilized to introduce the tank gas into the hollow part 148 smoothly.

Further, the gas inflow port 154 has an aperture plane that extends parallel to the direction B in which the first end 144 and the second end 146 are arranged, and the aperture plane is provided to intersect the direction of the gas flow F. This allows the gas flow F to hit the gas inflow port 154 directly so that the efficiency of introducing the gas subject to detection into the gas detection unit 101 is further increased.

In further accordance with this embodiment, the second end 146 is provided in an area where the tank gas flows downward in the vertical direction, and the gas inflow port 154 is provided above the gas outflow port 156 in the vertical direction. This further increases the aforementioned pressure difference by utilizing the gravity exerted on the tank gas. Accordingly, the efficiency of introducing the gas subject to detection into the gas detection unit 101 is further increased.

The aperture area of the gas inflow port 154 is larger than that of the gas outflow port 156. This creates a differential pressure in the first area 148a and the second area 148b due to the difference in aperture area (Bernoulli's principle). To be more specific, the pressure in the second area 148b will be lower than in the first area 148a. Therefore, the flow rate of the tank gas in the second area 148b will be higher than in the first area 148a. Accordingly, the efficiency of introducing the gas subject to detection into the gas detection unit 101 is further increased.

Further, the constant-temperature apparatus 1 according to this embodiment is further provided with a fan 6 that blows air to cause the tank gas to flow along the wall surface 4b. The gas sensor 100 is provided downstream of the fan 6 in the gas flow F. By providing the fan 6, the pressure difference between the gas inflow port 154 side and the gas outflow port 156 side is further increased. Accordingly, the efficiency of introducing the gas subject to detection into the gas detection unit 101 is further increased.

The constant-temperature apparatus 1 is further provided with a gas passage 8 in which the tank gas flows. The gas passage 8 is provided along the wall surface 4b in the internal space of the constant-temperature tank 4. For example, the gas passage 8 is defined by a partition plate 10 extending along the wall surface 4b and by the wall surface 4b. By providing the gas passage 8, it is further ensured that the gas flow F hits the second end 146 of the gas sensor 100. Accordingly, the efficiency of introducing the gas subject to detection into the gas detection unit 101 is further increased. In this embodiment, the fan 6 is provided near the entrance of the gas passage 8. Further, the constant-temperature apparatus 1 includes a gas introduction pipe 12 for introducing the tank gas into the constant-temperature tank 4. Preferably, the gas introduction pipe 12 is provided such that the fan 6 is positioned between the gas introduction pipe 12 and the gas sensor 100.

The operation of the gas sensor 100 to detect the density and humidity of the gas subject to detection is the same as that of embodiments 1-3. The following variations the gas sensor 100 according to embodiment 4 are possible.

### (Variation 3)

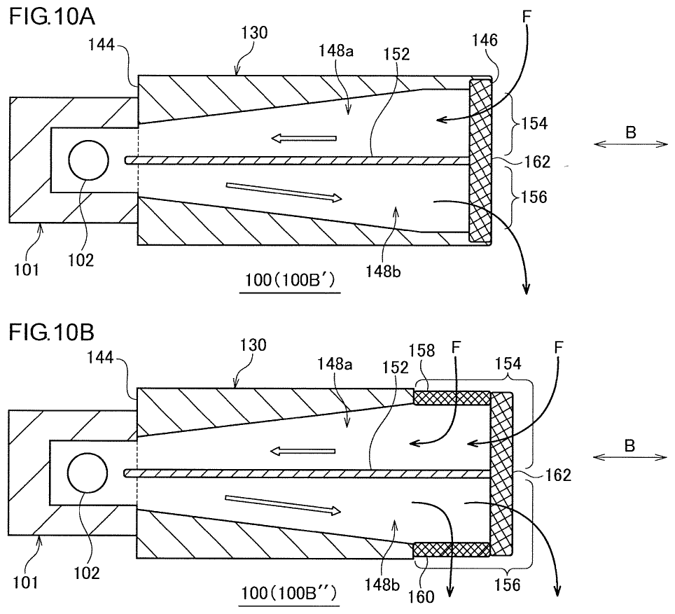

FIG. 10A is a vertical cross-sectional view schematically showing the gas sensor according to variation 3. Those features of the gas sensor according to this variation that are different from those of embodiment 4 will mainly be described. Common features will be described briefly, or a description thereof will be omitted. The aperture planes of the gas inflow port 154 and the gas outflow port 156 of the gas sensor 100 (100B′) according to variation 3 extend in a direction intersecting the direction B in which the first end 144 and the second end 146 are arranged. Therefore, the aperture planes of the gas inflow port 154 and the gas outflow port 156 extend substantially parallel to the gas flow F in the constant-temperature tank 4. The gas inflow port 154 and the gas outflow port 156 are blocked by a porous member 162 that allows passage of the gas subject to detection. The material for forming the porous member 162 is exemplified by the material to form the cap 150. The configuration also allows the tank gas to be introduced into the hollow part 148 by utilizing the pressure difference between the gas inflow port 154 side and the gas outflow port 156 side.

### (Variation 4)

FIG. 10B is a vertical cross-sectional view schematically showing the gas sensor according to variation 4. Those features of the gas sensor according to this variation that are different from those of embodiment 4 will mainly be described. Common features will be described briefly, or a description thereof will be omitted. The gas sensor 100 (100B″) according to variation 4 has a structure in which embodiment 4 and variation 1 are combined. In other words, the aperture planes of the gas inflow port 154 and the gas outflow port 156 have an area in which they extend parallel to the direction B in which the first end 144 and the second end 146 are arranged and an area in which they extend in a direction intersecting the direction B in which the first end 144 and the second end 146 are arranged. This increases the amount of tank gas introduced into the hollow part 148 so that the efficiency of introducing the gas subject to detection into the gas detection unit 101 is further increased.

### Embodiment 5

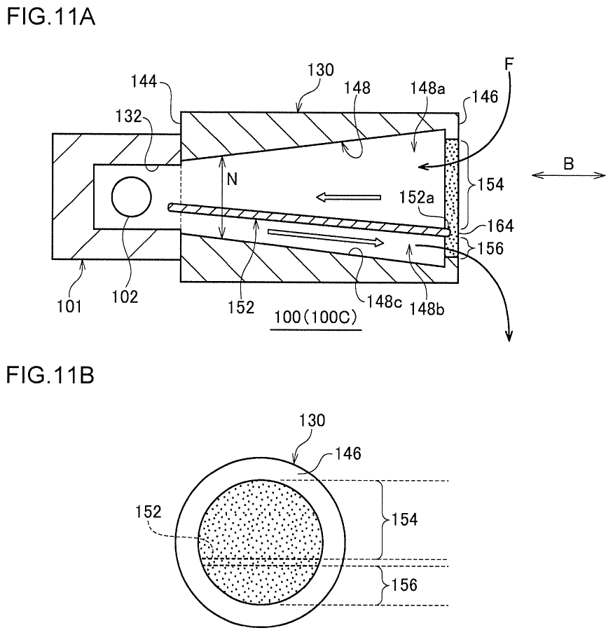

FIG. 11A is a vertical cross-sectional view schematically showing the gas sensor according to embodiment 5. FIG. 11B schematically shows the end face of the gas sensor according to embodiment 5 at the second end; In FIG. 11A, illustration of the interior of the gas detection unit 101 is simplified, and illustration of the temperature adjustment unit 180, the heat dissipating fin 182, the humidity detection unit 190, etc. is omitted. Those features of the gas sensor according to this embodiment that are different from those of embodiment 1 will mainly be described. Common features will be described briefly, or a description thereof will be omitted.

The gas passage 130 provided in the gas sensor 100 (100C) according to this embodiment includes a partition member 152. The partition member 152 divides the hollow part 148 into at least two areas including a first area 148a and a second area 148b. In this embodiment, the partition member 152 divides the hollow part 148 into two areas, i.e., the first area 148a and the second area 148b. Each of the first area 148a and the second area 148b extends from the first end 144 to the second end 146. Further, the hollow part 148 of the gas passage 130 is sloped such that a lower surface 148c in the vertical direction descends in the vertical direction away from the first end 144 and toward the second end 146.

Further, the gas passage 130 includes a gas inflow port 154 and a gas outflow port 156. The gas inflow port 154 is provided at the second end 146 and connects the internal space of the constant-temperature tank 4 to the first area 148a. The gas outflow port 156 is provided at the second end 146 and connects the second area 148b to the internal space of the constant-temperature tank 4. The gas inflow port 154 and the gas outflow port 156 are blocked by a porous member 164 that allows passage of the gas subject to detection. The material for forming the porous member 164 is exemplified by the material to form the cap 150. The aperture planes of the gas inflow port 154 and the gas outflow port 156 extend in a direction intersecting the direction B in which the first end 144 and the second end 146 are arranged.

The aperture area of the gas inflow port 154 is larger than that of the gas outflow port 156. In this embodiment, an end 152a of the partition member 152 toward the second end 146 is embedded in the porous member 164. The end 152a forms a boundary between the gas inflow port 154 and the gas outflow port 156. Further, the partition member 152 is sloped so as to descend in the vertical direction away from the first end 144 and toward the second end 146. This results in the formation of the gas inflow port 154 that has a larger aperture area and the gas outflow port 156 that has a smaller aperture area.

The gas sensor 100 is provided on the wall surface 4b of the constant-temperature tank 4 (see FIGS. 1 and 9A). The gas inflow port 154 is provided upstream in the gas flow F, and the gas outflow port 156 is provided downstream of the gas inflow port 154 in the gas flow F. Further, the second end 146 is provided in an area where the gas subject to detection flows downward in the vertical direction in the constant-temperature tank 4. Further, the gas inflow port 154 is provided above the gas outflow port 156 in the vertical direction.

The tank gas in the constant-temperature tank 4 flows into the hollow part 148 from the gas inflow port 154, flows in the first area 148a toward the first end 144, and arrives at the gas detection unit 101. In association with this, the tank gas located in the gas detection unit 101 flows in the second area 148b toward the second end 146 and flows out from the gas outflow port 156 into the constant-temperature tank 4. Thus, by using the partition member 152, the flow of the tank gas in the hollow part 148 is straightened. Accordingly, the speed of detection by the gas sensor 100 is improved.

The aperture area of the gas inflow port 154 is larger than that of the gas outflow port 156. This creates a differential pressure in the first area 148a and the second area 148b due to the difference in aperture area. Therefore, the flow rate of the tank gas in the second area 148b will be higher than in the first area 148a. Accordingly, the efficiency of introducing the gas subject to detection into the gas detection unit 101 is further increased.

The slope of the partition member 152 causes the cross-sectional area of the flow passage in the second area 148b to be smaller than the cross-sectional area of the flow passage in the first area 148a at least in part. Therefore, the flow rate of the tank gas in the second area 148b will be higher than in the first area 148a. Accordingly, the efficiency of introducing the gas subject to detection into the gas detection unit 101 is further increased.

In further accordance with this embodiment, the second end 146 is provided in an area where the tank gas flows downward in the vertical direction, and the gas inflow port 154 is provided above the gas outflow port 156 in the vertical direction. The feature further increases the pressure difference between the gas inflow port 154 and the gas outflow port 156 by utilizing the gravity exerted on the tank gas. Accordingly, the efficiency of introducing the gas subject to detection into the gas detection unit 101 is further increased.

The operation of the gas sensor 100 to detect the density and humidity of the gas subject to detection is the same as that of embodiments 1-3. The following variations of the gas sensor 100 according to embodiment 5 are possible.

### (Variation 5)

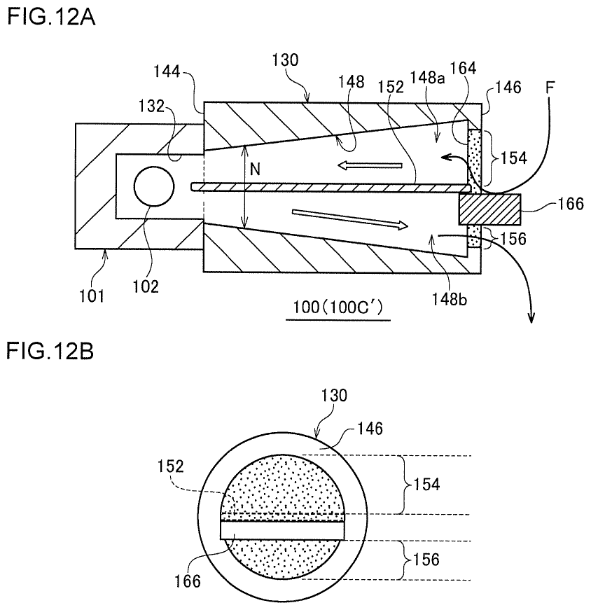

FIG. 12A is a vertical cross-sectional view schematically showing the gas sensor according to variation 5. FIG. 12B schematically shows the end face of the gas sensor according to variation 5 at the second end. Those features of the gas sensor according to this variation that are different from those of embodiment 5 will mainly be described. Common features will be described briefly, or a description thereof will be omitted. A difference in the aperture area is provided between the gas inflow port 154 and the gas outflow port 156 by blocking a portion of the gas outflow port 156 in the gas sensor 100 (100C′) according to variation 5. In this variation, a straightener 166 having a fin structure is used as a member to block a portion of the gas outflow port 156.

The straightener 166 projects from an area at the second end 146 between the gas inflow port 154 and the gas outflow port 156 into the internal space of the constant-temperature tank 4. The end of the straightener 166 toward the gas passage 130 is embedded in an area of the porous member 164 in contact with the second area 148b. This causes the aperture area of the gas outflow port 156 to be smaller than the aperture area of the gas inflow port 154.

The part of the straightener 166 projecting into the internal space of the constant-temperature tank 4 restricts the gas flow F (the flow of the gas subject to detection) in the constant-temperature tank 4. A portion of the tank gas flowing in the constant-temperature tank 4 hits the straightener 166 as it passes the neighborhood of the second end 146 and is guided to travel toward the gas inflow port 154. Therefore, the straightener 166 can guide the tank gas into the hollow part 148. This further increases the efficiency of introducing the gas subject to detection into the gas detection unit 101.

### Embodiment 6

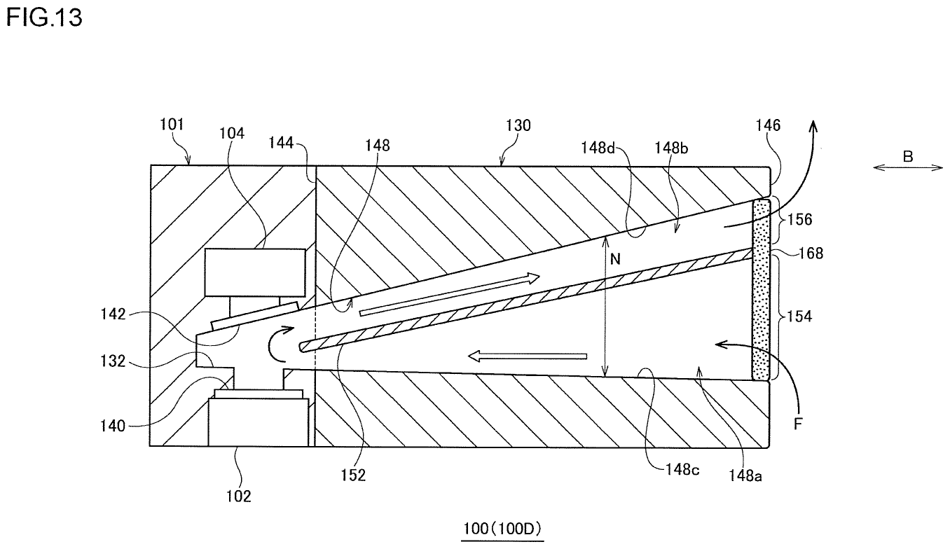

FIG. 13 is a vertical cross-sectional view schematically showing the gas sensor according to embodiment 6. In FIG. 13, illustration of the interior of the gas detection unit 101 is simplified, and illustration of the temperature adjustment unit 180, the heat dissipating fin 182, the humidity detection unit 190, etc. is omitted. Those features of the gas sensor according to this embodiment that are different from those of embodiment 1 will mainly be described. Common features will be described briefly, or a description thereof will be omitted.

The gas passage 130 provided in the gas sensor 100 (100D) according to this embodiment includes a partition member 152. The partition member 152 divides the hollow part 148 into at least two areas including a first area 148a and a second area 148b. In this embodiment, the partition member 152 divides the hollow part 148 into two areas, i.e., the first area 148a and the second area 148b. Each of the first area 148a and the second area 148b extends from the first end 144 to the second end 146. The hollow part 148 is sloped such that a lower surface 148c in the vertical direction descends in the vertical direction away from the first end 144 and toward the second end 146.

Further, the gas passage 130 includes a gas inflow port 154 and a gas outflow port 156. The gas inflow port 154 is provided at the second end 146 and connects the internal space of the constant-temperature tank 4 to the first area 148a. The gas outflow port 156 is provided at the second end 146 and connects the second area 148b to the internal space of the constant-temperature tank 4. The gas inflow port 154 and the gas outflow port 156 are blocked by a porous member 168 that allows passage of the gas subject to detection. The material for forming the porous member 168 is exemplified by the material to form the cap 150. The aperture planes of the gas inflow port 154 and the gas outflow port 156 extend in a direction intersecting the direction B in which the first end 144 and the second end 146 are arranged. The aperture area of the gas inflow port 154 is larger than that of the gas outflow port 156.

In this embodiment, the light source 102 is provided below the density detector 104 in the vertical direction. Further, the gas inflow port 154 is provided below the gas outflow port 156 in the vertical direction. Therefore, the first area 148a extends below the second area 148b in the vertical direction. In the gas detection unit 101, the heat of the light source 102 provided below in the vertical direction heats the gas in the gas introduction chamber 132. This creates a flow of gas that rises from the light source 102 toward the density detector 104. The gas turned into an upward flow in the gas introduction chamber 132 advances in the second area 148b toward the second end 146 and flows out from the gas outflow port 156. Meanwhile, the pressure is lowered in the first area 148a due to the upward flow of the gas caused by the heat of the light source 102. This causes the tank gas to flow into the first area 148a from the gas inflow port 154. This creates a tank gas circulation, in which the tank gas flowing into the hollow part 148 from the gas inflow port 154 arrives at the gas detection unit 101 and flows out from the gas outflow port 156 by flowing through the hollow part 148 again.

Thus, by using the partition member 152, the flow of the tank gas in the hollow part 148 is straightened. Accordingly, the speed of detection by the gas sensor 100 is improved. Since the gas subject to detection is circulated by using the heat of the light source 102, the speed of detection by the gas sensor 100 is further improved.

The hollow part 148 is tapered such that an upper surface 148d in the vertical direction inclines upward in the vertical direction away from the first end 144 and toward the second end 146. This makes the flow of the tank gas from the gas detection unit 101 to the gas outflow port 156 smoother. Accordingly, the speed of detection by the gas sensor 100 is improved. The slope of the upper surface 148d in the vertical direction is steeper than the slope of the lower surface 148c in the vertical direction. Accordingly, the size of the gas sensor 100 is prevented from growing.

The aperture area of the gas inflow port 154 is larger than that of the gas outflow port 156. This creates a differential pressure in the first area 148a and the second area 148b due to the difference in aperture area. Therefore, the flow rate of the tank gas in the second area 148b will be higher than in the first area 148a. Accordingly, the efficiency of introducing the gas subject to detection into the gas detection unit 101 is further increased. Further, the partition member 152 is tapered so as to ascend in the vertical direction away from the first end 144 and toward the second end 146. This causes the cross-sectional area of the flow passage in the second area 148b to be smaller than the cross-sectional area of the flow passage in the first area 148a at least in part. Therefore, the flow rate of the tank gas in the second area 148b will be higher than in the first area 148a. Accordingly, the efficiency of introducing the gas subject to detection into the gas detection unit 101 is further increased.

### Embodiment 7

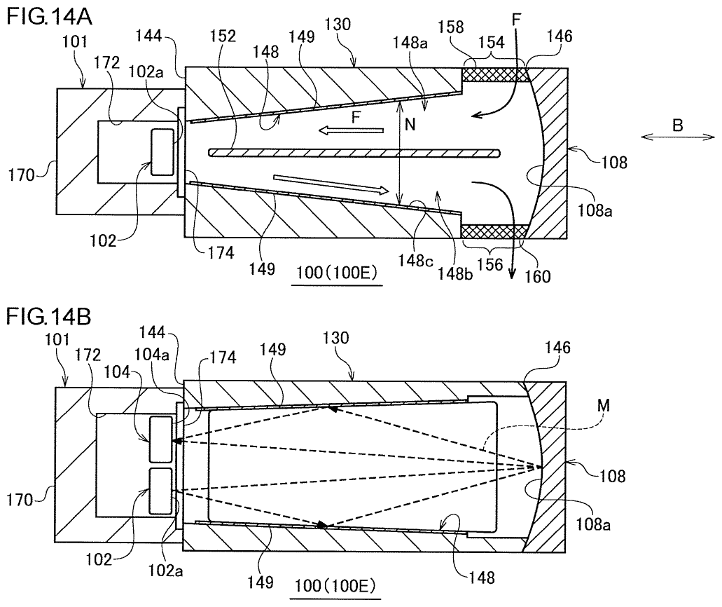

FIG. 14A is a vertical cross-sectional view schematically showing the gas sensor according to embodiment 7. FIG. 14B is a horizontal cross-sectional view schematically showing the gas sensor according to embodiment 7. In FIGS. 14A and 14B, illustration of the interior of the gas detection unit 101 is simplified, and illustration of the temperature adjustment unit 180, the heat dissipating fin 182, the humidity detection unit 190, etc. is omitted. Those features of the gas sensor according to this embodiment that are different from those of embodiment 1 mainly be described. Those features that are common to embodiment 1 will be described briefly, or a description thereof will be omitted.

The gas sensor 100 (100E) according to this embodiment is provided with the gas detection unit 101 including the light source 102 and the density detector 104, and with the gas passage 130. The gas passage 130 includes the first end 144, the second end 146, the hollow part 148, and the partition member 152. The first end 144 is provided toward the gas detection unit 101, and the second end 146 is provided toward the constant-temperature tank 4. The gas passage 130 circulates the gas subject to detection between the constant-temperature tank 4 and the gas detection unit 101 via the hollow part 148. The hollow part 148 has a shape in which the cross-sectional area N of the flow passage grows smaller away from the second end 146 and toward the first end 144. Further, the hollow part 148 is sloped such that a lower surface 148c in the vertical direction descends in the vertical direction away from the first end 144 and toward the second end 146.

The partition member 152 is a member that divides the hollow part 148 into at least two areas including a first area 148a and a second area 148b. In this embodiment, the partition member 152 divides the hollow part 148 into two areas, i.e., the first area 148a and the second area 148b. The ends of the partition member 152 do not reach the first end 144 and the second end 146. Therefore, the first area 148a and the second area 148b communicate with each other in the hollow part 148. The area of connection between the first area 148a and the second area 148b at the first end 144 and the area of connection at the second end 146 are configured such that a majority or the entirety of the tank gas flowing in from the gas inflow port 154 flows toward the first end 144 via the first area 148a.

Further, the gas passage 130 includes a gas inflow port 154 and a gas outflow port 156. The gas inflow port 154 is provided at the second end 146 and connects the constant-temperature tank 4 to the first area 148a. The gas outflow port 156 is provided at the second end 146 and connects the second area 148b to the constant-temperature tank 4. The gas inflow port 154 is blocked by a porous member 158, and the gas outflow port 156 is blocked by a porous member 160. The aperture planes of the gas inflow port 154 and the gas outflow port 156 extend parallel to a direction B in which the first end 144 and the second end 146 are arranged.

The light source 102 is provided such that the light emitting surface 102a faces the hollow part 148. In other words, the light source 102 is provided such that the emitted light M passes through the hollow part 148. Further, the density detector 104 is provided such that the light receiving surface 104a faces the hollow part 148. The relative positions of the light source 102 and the density detector 104 are defined such that the light M emitted from the light source 102 does not directly irradiate the light receiving surface 104a of the density detector 104.

Further, the gas sensor 100 is provided with a light reflecting part 108. The light reflecting part 108 is fixed to the second end 146 of the gas passage 130. The light reflecting part 108 includes a concave reflecting surface 108a. The concave reflecting surface 108a is provided to face the hollow part 148. This causes the concave reflecting surface 108a to be opposite to the light source 102 and the density detector 104. The concave reflecting surface 108a can be formed by, for example, forming a film of metal such as gold, aluminum, and chromium having a high reflectance in the infrared region on the surface of the light reflecting part 108.

The light M emitted from the light source 102 travels in the hollow part 148, is reflected by the concave reflecting surface 108a, travels in the hollow part 148 again, and arrives at the density detector 104. Therefore, the hollow part 148 also functions as a passage of the light M. A metal film 149 is formed on the wall surface defining the hollow part 148. The metal film 149 is made of, for example, gold, aluminum, chromium, etc. having a high reflectance in the infrared region. By providing the metal film 149, the light of the light source 102 is inhibited from being absorbed by the wall surface of the hollow part 148, and the efficiency of light guidance from the light source 102 to the density detector 104 is increased. As a result, the sensitivity of detection by the gas sensor 100 is increased.

Further, the partition member 152 is made of a metal. For example, the partition member 152 is made of gold, aluminum, chromium, etc. having a high reflectance in the infrared region. Further, the surface of the partition member 152 is preferably mirror-finished to increase the reflectance for the light M. This increases the efficiency of guiding the light from the light source 102 to the density detector 104.

The gas detection unit 101 includes a bracket 170. The bracket 170 includes a housing 172 for the light source 102 and the density detector 104. The housing 172 includes an opening toward the hollow part 148. The opening is blocked by a translucent member 174. Preferably, the opening in the housing 172 is hermetically sealed by the translucent member 174. The translucent member 174 is made of a material that transmits the light from the light source 102. In this embodiment, infrared light is emitted from the light source 102. Therefore, the translucent member 174 is made of, for example, germanium, silicon, sapphire, etc. The temperature adjustment unit 180 (see FIG. 1) varies the temperature of the translucent member 174. The first temperature sensor 184 (see FIG. 1) senses the temperature of the translucent member 174.

The gas sensor 100 is provided on the wall surface 4b of the constant-temperature tank 4 such that the gas inflow port 154 and the gas outflow port 156 project from the wall surface 4b of the constant-temperature tank 4 into the tank (see FIG. 9A). The gas inflow port 154 is provided such that the aperture plane intersects the gas flow F in the constant-temperature tank 4. Further, the gas outflow port 156 is provided opposite to the gas inflow port 154 in the direction of the gas flow F. Further, the gas inflow port 154 is provided upstream in the gas flow F, and the gas outflow port 156 is provided downstream of the gas inflow port 154 in the gas flow F. Further, the second end 146 is provided in an area where the gas subject to detection flows downward in the vertical direction in the constant-temperature tank 4. Further, the gas inflow port 154 is provided above the gas outflow port 156 in the vertical direction.

The tank gas located in the constant-temperature tank 4 flows into the hollow part 148 from the gas inflow port 154, flows in the first area 148a toward the first end 144, and arrives at the gas detection unit 101. In association with this, the tank gas located in the gas detection unit 101 flows in the second area 148b toward the second end 146 and flows out from the gas outflow port 156 into the constant-temperature tank 4.

Thus, by using the partition member 152, the flow of the tank gas in the hollow part 148 is straightened. This further increases the efficiency of introducing the gas subject to detection into the gas detection unit 101 and improves the speed of detection by the gas sensor 100.

By projecting the second end 146 of the gas sensor 100 into the area where the gas flow F is located, a pressure difference between the surface upstream in the gas flow F and the surface downstream is created. By providing the gas inflow port 154 on the upstream side in the gas flow F and providing the gas outflow port 156 on the downstream side, the pressure difference can be utilized to introduce the tank gas into the hollow part 148 smoothly.

Further, the gas inflow port 154 has an aperture plane that extends parallel to the direction B in which the first end 144 and the second end 146 are arranged, and the aperture plane is provided to intersect the direction of the gas flow F. This further increases the efficiency of introducing the gas subject to detection into the gas detection unit 101. Further, the second end 146 is provided in an area where the tank gas flows downward in the vertical direction, and the gas inflow port 154 is provided above the gas outflow port 156 in the vertical direction. This further increases the efficiency of introducing the gas subject to detection into the gas detection unit 101.

The aperture area of the gas inflow port 154 is larger than that of the gas outflow port 156. This creates a differential pressure in the first area 148a and the second area 148b due to the difference in aperture area. This further increases the efficiency of introducing the gas subject to detection into the gas detection unit 101.

The light M emitted from the light source 102 travels in the hollow part 148 and arrives at a second end 106b either directly or by being reflected by the metal film 149 and the partition member 152. The light M is reflected by the concave reflecting surface 108a, travels again in the hollow part 148, and arrives at the density detector 104 either directly or by being reflected by the metal film 149 and the partition member 152. In this process, the light M passes through the tank gas filling the hollow part 148. During the passage, the first light is absorbed by the gas subject to detection contained in the tank gas.

The density detector 104 detects the presence and density of the gas subject to detection based on the intensity of the first light in the light received by the density detector 104 with reference to the intensity of the first light in the light emitted from the light source 102. The temperature adjustment unit 180 varies the temperature of the translucent member 174 to induce dew concentration on the translucent member 174. Accordingly, the humidity detection unit 190 (see FIG. 1) is capable of acquiring the dew-point temperature c. The humidity detection unit 190 detects the humidity of the gas subject to detection by referring to the dew-point temperature c and the ambient temperature.

Thus, by causing the light M to pass through the hollow part 148 filled with the tank gas to detect the density and humidity of the gas subject to detection, the measurement distance of the gas subject to detection is increased. As a result, the gas that requires a relatively long measurement distance is detected with a higher accuracy. Even if the gas subject to detection is in a very small amount, the gas is detected with a high accuracy.

Preferably, the opening in the hollow part 148 at the first end 144 is elliptical, and the opening at the second end 146 is circular. Further, the hollow part 148 has a shape that changes progressively from elliptical to circular from the first end 144 to the second end 146. This improves the efficiency of transmission of the light M from the light source 102 to the density detector 104 as compared with a hollow part that is circular or elliptical at both ends.

Where the first end 144 of the hollow part 148 is configured to be elliptical, it is preferable that the light source 102 and the density detector 104 be arranged at positions point-symmetric with respect to the center of the ellipse (the point of intersection of the long axis and the short axis of the ellipse) when viewed in the direction in which the light source 102, the density detector 104, and the hollow part 148 are arranged. In this case, arbitrary parts of the light source 102 and the density detector 104 are provided at point-symmetric positions by way of example. Alternatively, the center of the light emitting surface 102a of the light source 102 and the center of the light receiving surface 104a of the density detector 104 are provided at point-symmetric positions. The feature allows the light emitted from the light source 102 to be focused on the density detector 104 more properly. The feature improves the efficiency of transmitting the light M. Further, the light source 102 and the density detector 104 are more preferably provided on the long diameter of the ellipse. Still more preferably, the light source 102 is provided on one focal point of the ellipse, and the density detector 104 is provided on the other focal point of the ellipse. The feature further improves the efficiency of transmitting the light M.

The embodiments and variations of the present invention are not limited to those described above and the embodiments and variations may be combined, or various further modifications such as design changes may be made based on the knowledge of a skilled person. The embodiments and variations resulting from such combinations or further modification are also within the scope of the present invention. New embodiments created by combinations of the above-described embodiments and variations and new embodiments created by further modifications to the embodiments and variations provide combined advantages of the embodiments, variations, and further modifications.

The constant-temperature apparatus 1 according to the embodiments and variations described above is exemplified by a CO₂ incubator but may be another apparatus so long as the constant-temperature apparatus 1 is provided with a constant-temperature tank 4 filled the gas subject to detection. The gas sensor 100 according to the embodiments and variations described above can be suitably used to measure the density and humidity of a gas in a high-temperature environment. For example, the gas sensor 100 can be used to measure an exhaust gas, combustion gas, etc.

The gas subject to detection may be a gas other than CO₂. Other gases subject to detection may include sulfur dioxide (SO₂, absorption wavelength: 7.3 μm, 7.35 μm), sulfur trioxide (SO₂, absorption wavelength: 7.25 μm, 7.14 μm), nitric monoxide (NO, absorption wavelength: 5.3 μm, 5.5 μm), carbon monoxide (CO, absorption wavelength: 4.2 μm), nitrogen monoxide (N₂O, absorption wavelength: 4 μm, 4.5 μm, 7.9 μm), nitrogen dioxide (NO₂, absorption wavelength, 5.7 μm, 6.3 μm), etc.

A variable wavelength filter in which the band of transmitted wavelength is variable may be provided in the density detector 104. The feature allows a single gas sensor 100 to detect a plurality of types of gas subject to detection.

Optional combinations of the aforementioned constituting elements, and implementations of the invention in the form of methods, apparatuses, and systems may also be practiced as additional modes of the present invention.

What is claimed is:

1. A gas sensor comprising:
a light source that emits a first light of a predetermined wavelength toward a gas subject to detection;
a density detector that receives the first light and detects a density of the gas subject to detection based on absorption of the first light by the gas subject to detection;
a translucent member provided between the light source and the density detector;
a temperature adjustment unit that varies a temperature of the translucent member;
a humidity detection unit that detects a humidity of the gas subject to detection based on variation in an amount of the first light received in the density detector and by using a temperature of the translucent member and an ambient temperature of the gas sensor; and
a gas passage that includes a first end, a second end opposite to the first end, and a hollow part extending from the first end to the second end, the light source, the density detector, and the translucent member being provided toward the first end, a gas space where the gas subject to detection is located being provided toward the second end, and the gas passage being configured to circulate the gas subject to detection between the first end and the second end via the hollow part, wherein
the hollow part has a shape in which a cross-sectional area of a flow passage grows smaller away from the second end and toward the first end either in steps or continuously,
the gas passage includes:
a partition member that divides the hollow part into at least two areas including a first area and a second area each extending from the first end to the second end, respectively;
a gas inflow port provided at the second end to connect the gas space to the first area; and
a gas outflow port provided at the second end to connect the second area to the gas space, wherein
the gas subject to detection located in the gas space flows from the gas inflow port into the hollow part, flows in the first area toward the first end, and arrives at a space between the light source and the density detector, and the gas subject to detection located in the space flows in the second area toward the second end and flows out from the gas outflow port to the gas space.

2. The gas sensor according to claim 1, wherein
the density detector includes:
a first detection unit; and
a first optical filter provided between the first detection unit and the light source and selectively transmitting the first light.

3. The gas sensor according to claim 1, wherein
the light source further emits a second light of a wavelength absorbed by water.

4. The gas sensor according to claim 3, wherein
the density detector further includes:
a second detection unit; and
a second optical filter provided between the second detection unit and the light source and selectively transmitting the second light.

5. The gas sensor according to claim 1, wherein
the light source further emits a third light of a wavelength not absorbed by the gas subject to detection or water.

6. The gas sensor according to claim 1, wherein
an aperture area of the gas inflow port is larger than that of the gas outflow port.

7. The gas sensor according to claim 1, wherein
the gas inflow port is provided upstream in a flow of the gas subject to detection in the gas space, and the gas outflow port is provided downstream of the gas inflow port in the flow of the gas subject to detection.

8. The gas sensor according to claim 7, wherein
the second end is provided in an area where the gas subject to detection flows downward in a vertical direction in the gas space, and
the gas inflow port is provided above the gas outflow port in the vertical direction.

9. The gas sensor according to claim 7, wherein
the gas inflow port has an aperture plane that extends parallel to a direction in which the first end and the second end are arranged, and the aperture plane is provided to intersect a direction in which the gas subject to detection flows in the gas space.

10. The gas sensor according to claim 1, further comprising:
a straightener that projects from an area at the second end between the gas inflow port and the gas outflow port into the gas space and restricts a flow of the gas subject to detection in the gas space.

11. The gas sensor according to claim 1, wherein
the gas passage is made of an adiabatic material at least in part.

12. The gas sensor according to claim 1, wherein
the light source and the density detector are provided such that a light emitting surface of the light source and a light receiving surface of the density detector face each other.

13. The gas sensor according to claim 1, wherein
the light source is provided below the density detector in the vertical direction, and
the gas inflow port is provided below the gas outflow port in the vertical direction.

14. The gas sensor according to claim 13, wherein
the hollow part is tapered such that an upper surface in the vertical direction inclines upward in the vertical direction away from the first end and toward the second end.

15. The gas sensor according to claim 14, wherein
the hollow part is tapered such that a lower surface in the vertical direction inclines downward in the vertical direction away from the first end and toward the second end, and
a slope of the upper surface in the vertical direction is steeper than a slope of the lower surface in the vertical direction.

16. The gas sensor according to claim 1, further comprising:
a gas introduction chamber provided between the light source and the density detector, the gas subject to detection flowing in through the gas introduction chamber;
a first housing provided adjacent to the gas introduction chamber and housing the light source;
a second housing provided adjacent to the gas introduction chamber and housing the density detector, wherein
the translucent member includes: a first translucent member that spaces the gas introduction chamber apart from the first housing; and a second translucent member that spaces the gas introduction chamber apart from the second housing.

17. The gas sensor according to claim 16, wherein
the density detector and the second translucent member are spaced apart from each other.

18. A constant-temperature apparatus comprising:
a constant-temperature tank that houses a gas; and
the gas sensor according to claim 1, wherein
the gas sensor detects a density and humidity of the gas in the constant-temperature tank.

19. The constant-temperature apparatus according to claim 18, wherein
the gas sensor is provided on a wall surface of the constant-temperature tank, and
the constant-temperature apparatus further comprises a fan that blows air to cause the gas to flow along the wall surface.

20. The constant-temperature apparatus according to claim 18, wherein
the gas sensor is provided on a wall surface of the constant-temperature tank, and
the constant-temperature apparatus further comprises a gas passage provided along the wall surface, the gas flowing in the gas passage.
